(19) United States
(12) Patent Application Publication
(10) Pub. No.: US 2024/0001215 A1
(43) Pub. Date: Jan. 4, 2024

(54) EXERCISE PROMOTION SYSTEM, CONTROL METHOD, AND COMPUTER READABLE MEDIUM

(71) Applicant: TOYOTA JIDOSHA KABUSHIKI KAISHA, Toyota-shi (JP)

(72) Inventors: Issei NAKASHIMA, Katsushika-ku (JP); Toru MIYAGAWA, Seto-shi (JP); Hideo HASEGAWA, Nagoya-shi (JP); Yusuke KINOSHITA, Ota-ku (JP); Kevin SCHAEFER, Kawasaki-shi (JP); Florin BAIDUC, Chiba-shi (JP); Ryota NAKABAYASHI, Nagoya-shi (JP); Joan CHEN, Chuo-ku (JP); Maxwell FORREST, Kawasaki-shi (JP)

(73) Assignee: TOYOTA JIDOSHA KABUSHIKI KAISHA, Toyota-shi (JP)

(21) Appl. No.: 18/346,238

(22) Filed: Jul. 2, 2023

(30) Foreign Application Priority Data
Jul. 4, 2022 (JP) 2022-107677

(51) Int. Cl. A63B 71/06; A61B 5/11

(52) U.S. Cl. CPC A63B 71/0622; A61B 5/1118; A61B 2503/24

(57) ABSTRACT

An exercise promotion system includes a work environment providing device configured to provide a work environment for improving efficiency of work of a user, an exercise state detection unit configured to detect an exercise state of the user's lower limb, and a work environment change unit configured to change the work environment provided by the work environment providing device. The work environment change unit changes the work environment so that the work environment deteriorates when the exercise state detected by the working state detection unit does not meet a predetermined reference.

### CROSS REFERENCE TO RELATED APPLICATIONS

This application is based upon and claims the benefit of priority from Japanese patent application No. 2022-107677, filed on Jul. 4, 2022, the disclosure of which is incorporated herein in its entirety by reference.

### BACKGROUND

The present disclosure relates to an exercise promotion system, a control method, and a computer readable medium.

Japanese Unexamined Patent Application Publication No. 2022-65360 discloses a pedaling exercise device.

### SUMMARY

In order to overcome the problem of a lack of daily exercise associated with office work, it is typically preferable to install a lower limb exercise device under a desk. That is, if lower limb exercises are performed using the lower limb exercise device while sitting at a desk during office work time, it would be possible to overcome the problem of a lack of daily exercise without bothering to set aside special time for exercise.

However, simply placing the lower limb exercise device under the desk does not mean that everyone will perform lower limb exercises.

Therefore, an object of the present disclosure is to provide a technique for motivating a user to perform lower limb exercises while working.

According to a first aspect of the present disclosure, an exercise promotion system including: a work environment providing device configured to provide a work environment for improving efficiency of work of a user; an exercise state detection unit configured to detect an exercise state of the user's lower limb; and a work environment change unit configured to change the work environment provided by the work environment providing device, in which the work environment change unit changes the work environment so that the work environment deteriorates when the exercise state detected by the working state detection unit does not meet a predetermined reference, is provided. According to the above configuration, it is possible to motivate the user to perform lower limb exercises while working.

After the work environment change unit changes the work environment so that the work environment deteriorates, the work environment change unit may change the work environment so that the work environment improves when the exercise state detected by the exercise state detection unit satisfies the predetermined reference. According to the above configuration, it is possible to further motivate the user to perform lower limb exercises while working.

A failure of the exercise state to meet the predetermined reference may mean that the user's lower limb is not exercised.

A failure of the exercise state to meet the predetermined reference may mean that the user's lower limb has not been exercised for more than a predetermined period of time.

A failure of the exercise state to meet the predetermined reference may mean that an exercise load of the user's lower limb falls below a predetermined reference.

The work environment providing device may be a desk, and the work environment change unit may change a state of a top plate of the desk so that the state of the top plate of the desk becomes a less appropriate state when the exercise state detected by the exercise state detection unit does not satisfy a predetermined reference.

The work environment providing device may be a chair, and the work environment change unit may change a state of a seat surface of the chair so that the state of the seat surface of the chair becomes a less appropriate state when the exercise state detected by the exercise state detection unit does not satisfy a predetermined reference.

The work environment providing device may be a display, and the work environment change unit may change a display state of the display so that the display state of the display becomes a less appropriate state when the exercise state detected by the exercise state detection unit does not satisfy a predetermined reference.

The display state of the display may indicate at least one of a height, an elevation angle, a horizontal angle, an effective region, brightness, and a contrast ratio of an image display surface of the display.

According to a second aspect of the present disclosure, a method of controlling an exercise promotion system including a work environment providing device configured to provide a work environment for improving efficiency of work of a user is provided. The method includes: detecting, by the exercise promotion system, an exercise state of the user's lower limb; and changing, by the exercise promotion system, the work environment so that the work environment deteriorates when the exercise state does not meet a predetermined reference.

According to the above method, it is possible to motivate the user to perform lower limb exercises while working.

A program that causes a computer to perform the above method is provided.

According to the present disclosure, it is possible to motivate a user to perform lower limb exercises while working.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a side view of a pedal exercise system;

FIG. 2 is a functional block diagram of an exercise promotion apparatus;

FIG. 3 is an operation flow of the exercise promotion apparatus;

FIG. 4 is a side view of the pedal exercise system when a top plate of a desk is raised;

FIG. 5 is a side view of the pedal exercise system when an image display surface of a display is raised;

FIG. 6 is a side view of the pedal exercise system when a seat surface of a chair is lowered;

FIG. 7 is an example of an appropriate display of the image display surface of the display;

FIG. 8 is an example of a display of the image display surface of the display with a reduced effective region;

FIG. 9 is an example of the display of the image display surface of the display with enhanced brightness; and

FIG. 10 is an example of the display of the image display surface of the display with a reduced contrast ratio.

### DESCRIPTION OF EMBODIMENTS

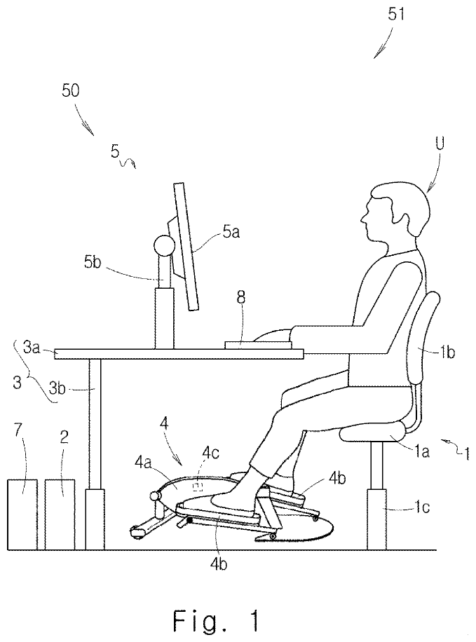

An embodiment of the present disclosure will be described below with reference to FIGS. 1 to 10.

FIG. 1 shows a user U seated on a chair 1 doing office work using a desktop computer 2. A pedaling exercise device 4 is installed under the desk 3. With this configuration, the user U can perform a pedal exercise using the pedaling exercise device 4 during office work. The pedaling exercise device 4 is a specific example of the lower limb exercise device. As the lower limb exercise device, a stepping exercise device can be considered in addition to the pedaling exercise device 4.

The desk 3 includes a top plate 3a and a lifting apparatus 3b. The lifting apparatus 3b supports the top plate 3a and moves up and down the top plate 3a based on an external command. The lifting apparatus 3b is typically composed of a linear actuator.

The chair 1 includes a seat surface 1a, a backrest 1b, and a lifting apparatus 1c. The lifting apparatus 1c supports the seat surface 1a and the backrest 1b, and moves up and down the seat surface 1a and the backrest 1b based on an external command. The lifting apparatus 1c is typically composed of a linear actuator.

A liquid crystal display 5 is installed on the top plate 3a of the desk 3. The liquid crystal display 5 has an image display surface 5a and a support mechanism 5b. The support mechanism 5b supports the image display surface 5a and adjusts a height, an elevation angle, and a horizontal angle of the image display surface 5a based on an external command. Here, the height of the image display surface 5a of the liquid crystal display 5 is typically the height relative to a line of sight of the user U. The elevation angle of the image display surface 5a of the liquid crystal display 5 is the angle formed by a virtual line perpendicular to the image display surface 5a of the liquid crystal display 5 and a horizontal line. The horizontal angle of the image display surface 5a of the liquid crystal display 5 is an orientation specified in plan view of a virtual line perpendicular to the image display surface 5a of the liquid crystal display 5. The support mechanism 5b is typically composed of a linear actuator or a rotary actuator.

An exercise promotion apparatus 7 is installed behind the desktop computer 2. The exercise promotion apparatus 7 is installed to motivate the user U to perform moderate pedal exercises using the pedaling exercise device 4 during office work.

The pedaling exercise device 4 has a pair of pedals 4b supported by an exercise device body 4a. The pair of pedals 4b are rotatably supported by a pair of crank arms. The pair of crank arms are connected to a crankshaft rotatably supported by the exercise device body 4a. Thus, the pair of pedals 4b are interlocked with each other. The exercise device body 4a is equipped with a loading apparatus that applies a load to the rotation of the crankshaft. The loading apparatus is typically an electromagnetic brake. The exercise device body 4a further has a crank angle sensor 4c that detects a crank angle of the crankshaft. The crank angle sensor 4c transmits crank angle data indicating the detected crank angle to the exercise promotion apparatus 7.

The support mechanism 5b, the lifting apparatus 1c, and the lifting apparatus 3b are electrically connected to the exercise promotion apparatus 7. The exercise promotion apparatus 7 controls operations of the support mechanism 5b, the lifting apparatus 1c, and the lifting apparatus 3b by outputting control signals to the support mechanism 5b, the lifting apparatus 1c, and the lifting apparatus 3b.

The chair 1, the desk 3, and the liquid crystal display 5 are installed to improve efficiency of the work of the user U. That is, the chair 1, the desk 3, and the liquid crystal display 5 assist the work of the user U. Therefore, the chair 1, the desk 3, and the liquid crystal display 5 provide a work environment for improving the efficiency of the work of the user U. The work environment provided by the chair 1 is to achieve a sitting position of user U. The work environment provided by the desk 3 is to support an input device such as a keyboard 8 used by the user U at an appropriate height. The work environment provided by the liquid crystal display 5 is to achieve an effortless head posture and image display with less visual burden.

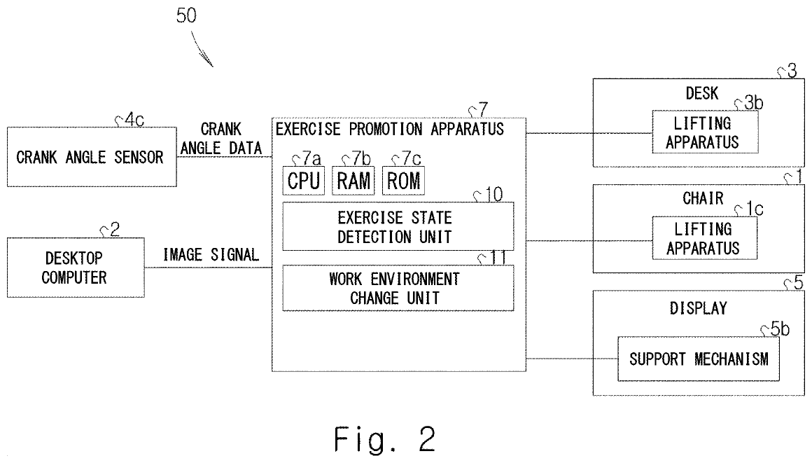

FIG. 2 shows the functional block diagram of the exercise promotion apparatus 7. As shown in FIG. 2, the exercise promotion apparatus 7 includes a CPU (Central Processing Unit) 7a, a RAM (Random Access Memory) 7b that can be freely read and written, and a ROM (Read-Only Memory) 7c. The CPU 7a reads and executes a control program stored in the ROM 7c, and the control program causes hardware such as the CPU 7a to function as an exercise state detection unit 10 and a work environment change unit 11.

The exercise promotion apparatus 7 receives the crank angle data, for example, every second from the crank angle sensor 4c of the pedaling exercise device 4. The exercise promotion apparatus 7 receives image signals from the desktop computer 2. The exercise promotion apparatus 7 also outputs the received image signals to the liquid crystal display 5.

The exercise state detection unit 10 detects an exercise state of the user U based on the crank angle data received from the crank angle sensor 4c of the pedaling exercise device 4. The exercise state may include the presence or absence of an exercise by the user U using the pedaling exercise device 4, a load of the exercise by the user U using the pedaling exercise device 4, and any other state relating to the exercise performed by the user U using the pedaling exercise device 4.

The work environment change unit 11 changes the work environment provided by the chair 1, the desk 3, and the liquid crystal display 5. Specifically, the work environment change unit 11 changes the work environment so that if the exercise state detected by the exercise state detection unit 10 does not satisfy a predetermined reference, the work environment deteriorates. After the work environment change unit 11 changes the work environment so that the work environment deteriorates, if the exercise state detected by the exercise state detection unit 10 satisfies the predetermined reference, it changes the work environment so that the work environment improves. According to the above configuration, if the exercise state does not satisfy the predetermined reference, since the work environment of office work deteriorates, which makes office work difficult, the user U will continue the pedal exercise using the pedaling exercise device 4 or resume the pedal exercise using the pedaling exercise device 4 so that the work environment of office work does not deteriorate. In this way, the user U will be motivated to exercise in parallel with office work, and thus the original object of overcoming the problem of a lack of daily exercise can be achieved.

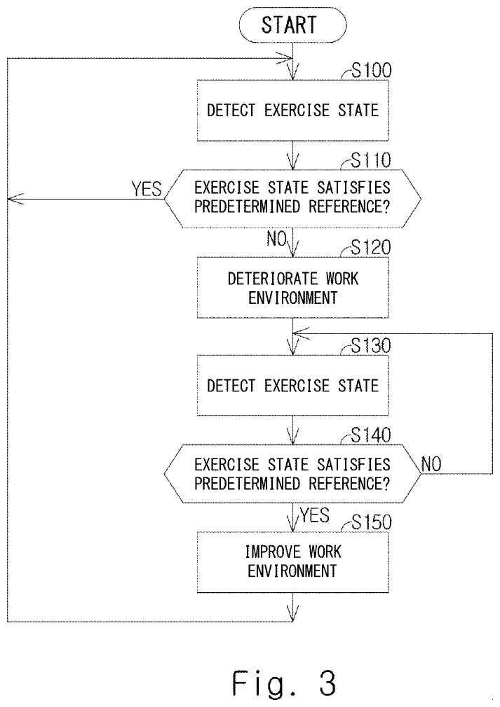

Next, an operation of the exercise promotion apparatus 7 will be described with reference to FIGS. 3 to 10. FIG. 3 shows an operation flow of the exercise promotion apparatus 7.

### S100:

First, the exercise state detection unit 10 detects the exercise state of the user U's lower limb based on the crank angle data received from the crank angle sensor 4c of the pedaling exercise device 4.

### S110:

Next, the work environment change unit 11 determines whether or not the exercise state of the user U's lower limb detected by the exercise state detection unit 10 satisfies the predetermined reference. If the exercise state of the user U's lower limb detected by the exercise state detection unit 10 satisfies the predetermined reference (S110: YES), the work environment change unit 11 returns the processing to S100. On the other hand, if the exercise state of the user U's lower limb detected by the exercise state detection unit 10 does not satisfy the predetermined reference (S110: NO), the work environment change unit 11 proceeds the processing to S120.

For example, if the work environment change unit 11 can detect that the user U is exercising using the pedaling exercise device 4, it determines that the exercise state of the user U's lower limb detected by the exercise state detection unit 10 satisfies the predetermined reference, whereas if the work environment change unit 11 cannot detect that the user U is exercising using the pedaling exercise device 4, it determines that the exercise state of the user U's lower limb detected by the exercise state detection unit 10 does not satisfy the predetermined reference.

Furthermore, if the work environment change unit 11 can detect that the user U is exercising using the pedaling exercise device 4, it determines that the exercise state of the user U's lower limb detected by the exercise state detection unit 10 satisfies the predetermined reference, whereas if the work environment change unit 11 cannot detect that the user U is continuing to exercise using the pedaling exercise device 4 for a predetermined period of time, it determines that the exercise state of the user U's lower limb detected by the exercise state detection unit 10 does not satisfy the predetermined reference.

In addition, for example, if a crank speed of the pedaling exercise device 4 exceeds a predetermined speed, the work environment change unit 11 determines that the exercise state of the user U's lower limb detected by the exercise state detection unit 10 satisfies the predetermined reference, whereas if the crank speed of the pedaling exercise device 4 falls below the predetermined speed, the work environment change unit 11 determines that the exercise state of the user U's lower limb detected by the exercise state detection unit 10 does not satisfy the predetermined reference.

As described above, the “predetermined reference” is used to determine whether or not the user U is exercising using the pedaling exercise device 4 without skipping. That is, if the user U is exercising using the pedaling exercise device 4 without skipping, the predetermined reference is considered to be satisfied.

### S120:

Next, the work environment change unit 11 changes the work environment so that the work environment deteriorates.

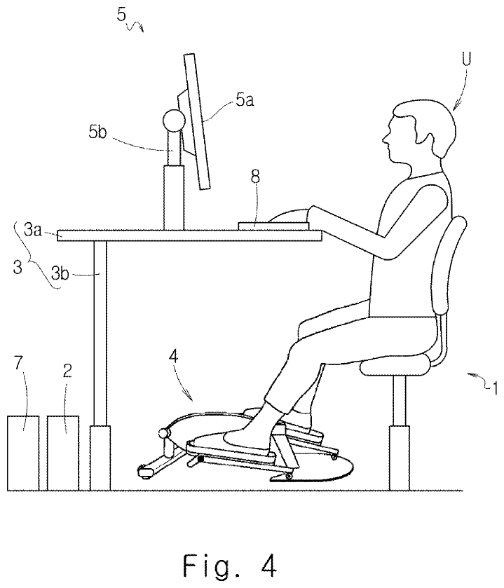

FIG. 4 shows how the work environment change unit 11 deteriorates the work environment. In the case of FIG. 4, the work environment change unit 11 transmits a control command to the lifting apparatus 3b of the desk 3 such that the top plate 3a is raised by 10 centimeters. The lifting apparatus 3b of the desk 3 raises the top plate 3a of the desk 3 by 10 centimeters based on the received control command. As a result, the top plate 3a of the desk 3 is raised, and consequently, the work environment deteriorates. That is, in FIG. 4, the height of the top plate 3a of the desk 3 is 10 centimeters higher than an appropriate height.

In this case, since the user U has to push both elbows forward, the muscles around the shoulder, such as the deltoid muscle, tend to get tired. At the same time, the height of the image display surface 5a of the liquid crystal display 5 becomes higher than the appropriate height, and thus the line of sight is inclined upward, which induces dry eyes, and the head is tilted backward, which causes the trapezius muscle to become easily tired. Also, since the user U's head does not fit within the viewing angle of the image display surface 5a of the liquid crystal display 5, the actual contrast ratio of the image display surface 5a deteriorates.

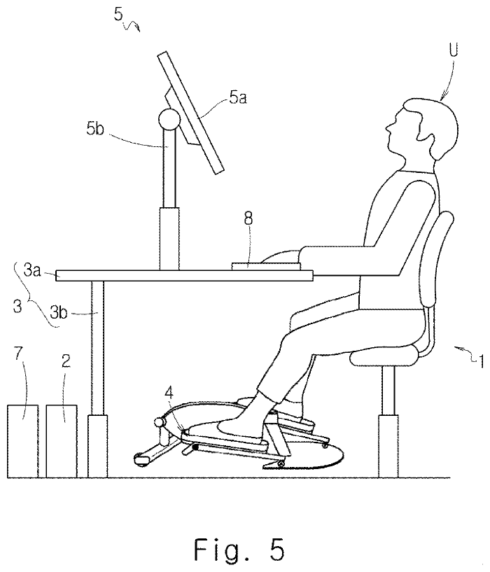

FIG. 5 shows how the work environment change unit 11 deteriorates the work environment. In the example of FIG. 5, the work environment change unit 11 transmits a control command to the support mechanism 5b of the liquid crystal display such that the image display surface 5a of the liquid crystal display 5 is raised by 10 centimeters and the image display surface 5a is directed upward by a predetermined angle. Based on the received control command, the support mechanism 5b of the liquid crystal display 5 raises the image display surface 5a of the liquid crystal display 5 by 10 centimeters and causes the image display surface 5a to be directed upward by a predetermined angle. As a result, the image display surface 5a of the liquid crystal display 5 is directed upward while the image display surface 5a is raised, and as a result, the work environment deteriorates. That is, in FIG. 5, the height of the image display surface 5a of the liquid crystal display 5 becomes 10 centimeters higher than the appropriate height, and the elevation angle of the image display surface 5a increases upward by a predetermined angle.

In this case, since the height of the image display surface 5a of the liquid crystal display 5 becomes higher than the appropriate height, the line of sight is inclined upward, which induces dry eyes, and the head is tiled backward, which causes the trapezius muscle to become easily tired. Also, since the user U's head does not fit within the viewing angle of the image display surface 5a of the liquid crystal display 5, the actual contrast ratio of the image display surface 5a deteriorates.

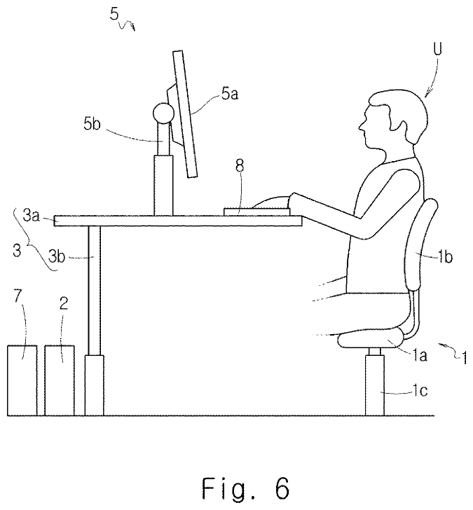

FIG. 6 shows how the work environment change unit 11 deteriorates the work environment. In the example of FIG. 6, the work environment change unit 11 transmits a control command to the lifting apparatus 1c of the chair 1 such that the seat surface 1a of the chair 1 is lowered by 10 centimeters. The lifting apparatus 1c of the chair 1 lowers the seat surface 1a of the chair 1 by 10 centimeters based on the received control command. As a result, the seat surface 1a of the chair 1 is lowered, and thus the work environment deteriorates. That is, in FIG. 6, the height of the seat surface 1a of the chair 1 is lower than an appropriate height by 10 centimeters.

In this case, since the user U has to push both elbows forward, the muscles around the shoulder, such as the deltoid muscle, tend to get tired. At the same time, the height of the image display surface 5a of the liquid crystal display 5 becomes higher than the appropriate height, and thus the line of sight is inclined upward, which induces dry eyes, and the head is tilted backward, which causes the trapezius muscle to become easily tired. Also, since the user U's head does not fit within the viewing angle of the image display surface 5a of the liquid crystal display 5, the actual contrast ratio of the image display surface 5a deteriorates.

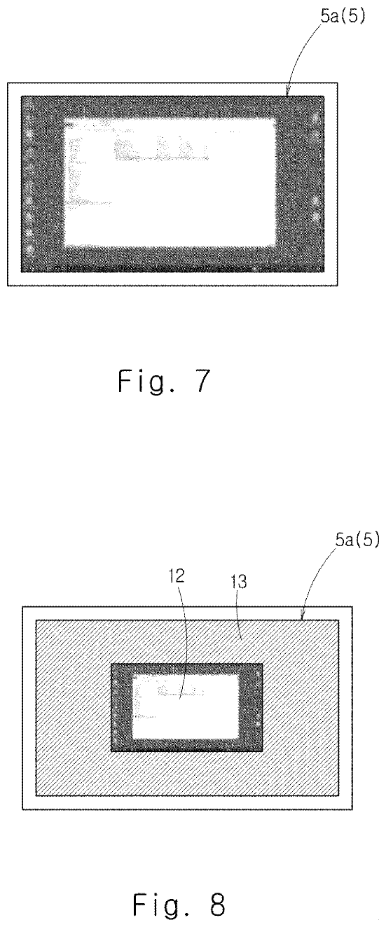

FIG. 7 shows an example of appropriate display of the image display surface 5a of the liquid crystal display 5. In the example of display shown in FIG. 7, the entire image display surface 5a of the liquid crystal display 5 is an effective region, and the image is displayed on the entire image display surface 5a. In addition, the brightness and the contrast ratio of the image display surface 5a of the liquid crystal display 5 are also at appropriate values.

On the other hand, FIGS. 8 to 10 show an example in which the work environment change unit 11 processes the image signal received from the desktop computer 2 and then outputs it to the liquid crystal display 5, thereby deteriorating the work environment related to the liquid crystal display 5.

In FIG. 8, only a narrow area 12 in the center of the image display surface 5a of the liquid crystal display 5 is defined as an effective region, and another area 13 is defined as an invalid region. That is, in FIG. 8, an image that should be originally displayed on the entire image display surface 5a is reduced and displayed in the area 12. In this case, since the text displayed in the area 12 of the image display surface 5a becomes smaller, the eyes become easily tired.

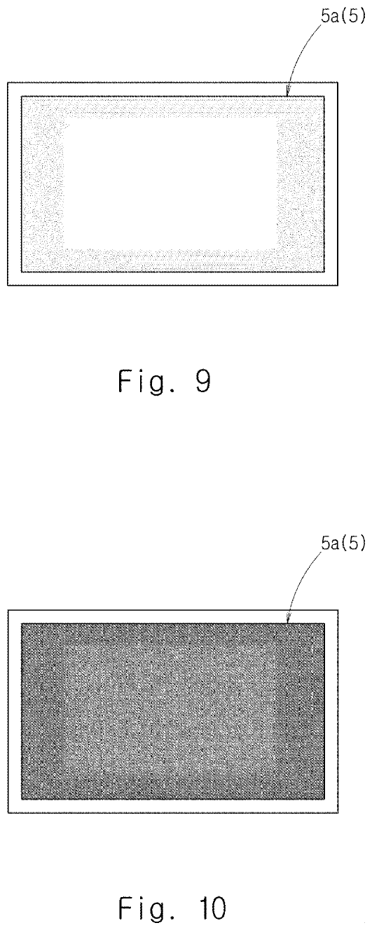

In FIG. 9, the brightness of the image display surface 5a of the liquid crystal display 5 is made higher than appropriate brightness. In this case, the eyes become easily tired from the dazzle.

In FIG. 10, the contrast ratio of the image display surface 5a of the liquid crystal display 5 is made lower than an appropriate contrast ratio. In this case, since the text displayed on the image display surface 5a becomes difficult to read, the eyes become easily tired.

### S130:

Returning to FIG. 3, the exercise state detection unit 10 detects the exercise state of the user U's lower limb based on the crank angle data received from the exercise device body 4.

### S140:

Next, the work environment change unit 11 determines whether or not the exercise state of the user U's lower limb detected by the exercise state detection unit 10 satisfies the predetermined reference. If the exercise state of the user U's lower limb detected by the exercise state detection unit 10 satisfies the predetermined reference (S140: YES), the work environment change unit 11 proceeds the processing to S150. On the other hand, if the exercise state of the user U detected by the exercise state detection unit 10 does not satisfy the predetermined reference (S140: NO), the work environment change unit 11 returns the processing to S130.

### S150:

Next, the work environment change unit 11 changes the work environment so that the work environment improves.

That is, in the case of FIG. 4, the work environment change unit 11 transmits a control command to the lifting apparatus 3b of the desk 3 such that the top plate 3a is lowered by 10 centimeters.

In the case of FIG. 5, the work environment change unit 11 transmits a control command to the support mechanism 5b of the liquid crystal display 5 such that the image display surface 5a of the liquid crystal display 5 is lowered by 10 centimeters and the image display surface 5a is directed downward by a predetermined angle.

In the case of FIG. 6, the work environment change unit 11 transmits a control command to the lifting apparatus 1c of the chair 1 such that the seat surface 1a of the chair 1 is raised by 10 centimeters.

In the case of FIGS. 8 to 10, the image signal received from the desktop computer 2 is output to the liquid crystal display 5 without any processing.

Thus, a suitable work environment for continuing the work is restored, thereby enabling the user U to concentrate on the work and overcome the problem of a lack of daily exercise.

The chair 1, the desk 3, and the liquid crystal display 5 are specific examples of the work environment providing device. As shown in FIG. 2, the work environment providing device, the exercise state detection unit 10, and the work environment change unit 11 constitute the exercise promotion system 50. The lower limb exercise system 51 shown in FIG. 1 includes the exercise promotion system 50 and the pedaling exercise device 4.

Although the embodiment of the present disclosure has been described above, the above embodiment has the following features.

That is, the exercise promotion system 50 includes the work environment providing device (e.g., desk 3) that provides a work environment for the user U to work efficiently, the exercise state detection unit 10 that detects the exercise state of the user U's lower limb, and the work environment change unit 11 that changes the work environment provided by the work environment providing device. If the exercise state detected by the exercise state detection unit 10 does not meet the predetermined reference, the work environment change unit 11 changes the work environment so that the work environment deteriorates. According to the above configuration, it is possible to motivate the user to do lower limb exercises while working.

In addition, after the work environment change unit 11 changes the work environment so that the work environment deteriorates, if the exercise state detected by the exercise state detection unit 10 satisfies the predetermined reference, it changes the work environment so that the work environment improves. According to the above configuration, it is further possible to motivate the user to do lower limb exercises while working.

In addition, a failure of the exercise state to meet the predetermined reference means that the user U's lower limb has not exercised, that the user's lower limb has not moved for more than a predetermined period of time, or that an exercise load on the user's lower limb falls below a predetermined reference.

If the exercise state detected by the exercise state detection unit does not meet the predetermined reference, the work environment change unit 11 may change the state of the top plate of the desk so that the state of the top plate of the desk becomes a less appropriate state.

When the work environment providing device is the chair 1, the work environment change unit 11 changes the state of the seat surface 1a of the chair 1 so that the state of the seat surface 1a of the chair 1 becomes a less appropriate state if the exercise state detected by the exercise state detection unit 10 does not meet a predetermined reference. In this case, the work environment change unit 11 typically causes the height of the seat surface 1a of the chair 1 to be higher or lower than the appropriate height. The work environment change unit 11 may cause a pitch attitude of the seat surface 1a of the chair 1 to lean forward or backward than an appropriate attitude. The work environment change unit 11 may cause a roll attitude of the seat surface 1a of the chair 1 to lean right or left than an appropriate attitude.

When the work environment providing device is the liquid crystal display 5, the work environment change unit 11 changes a display state of the display so that the display state of the liquid crystal display 5 becomes a less appropriate state if the exercise state detected by the exercise state detection unit 10 does not satisfy the predetermined reference. The display state of the liquid crystal display 5 is at least one of the height, the elevation angle, the horizontal angle, the effective region, the brightness, and the contrast ratio of the image display surface 5a of the liquid crystal display 5. The support mechanism 5b of the liquid crystal display 5 can increase or decrease the height, the elevation angle, and the horizontal angle of the image display surface 5a of the liquid crystal display 5.

In addition, the method of controlling the exercise promotion system 50 equipped with the work environment providing device to provide the work environment for improving the efficiency of the work of the user U includes a step in which the exercise promotion apparatus 7 of the exercise promotion system 50 detects the exercise state of the user U's lower limb (S100) and a step in which the work environment is changed so that the work environment deteriorates if the exercise state does not meet the predetermined reference (S120). According to the above method, it is possible to motivate the user to perform lower limb exercises in parallel with the work.

The programs can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. An exercise promotion system comprising:
a work environment providing device configured to provide a work environment for improving efficiency of work of a user;
an exercise state detection unit configured to detect an exercise state of the user's lower limb; and
a work environment change unit configured to change the work environment provided by the work environment providing device, wherein
the work environment change unit changes the work environment so that the work environment deteriorates when the exercise state detected by the working state detection unit does not meet a predetermined reference.

2. The exercise promotion system according to claim 1, wherein
after the work environment change unit changes the work environment so that the work environment deteriorates, the work environment change unit changes the work environment so that the work environment improves when the exercise state detected by the exercise state detection unit satisfies the predetermined reference.

3. The exercise promotion system according to claim 1, wherein
a failure of the exercise state to meet the predetermined reference means that the user's lower limb is not exercised.

4. The exercise promotion system according to claim 1, wherein
a failure of the exercise state to meet the predetermined reference means that the user's lower limb has not been exercised for more than a predetermined period of time.

5. The exercise promotion system according to claim 1, wherein
a failure of the exercise state to meet the predetermined reference means that an exercise load of the user's lower limb falls below a predetermined reference.

6. The exercise promotion system according to claim 1, wherein
the work environment providing device is a desk, and
the work environment change unit changes a state of a top plate of the desk so that the state of the top plate of the desk becomes a less appropriate state when the exercise state detected by the exercise state detection unit does not satisfy a predetermined reference.

7. The exercise promotion system according to claim 1, wherein
the work environment providing device is a chair, and
the work environment change unit changes a state of a seat surface of the chair so that the state of the seat surface of the chair becomes a less appropriate state when the exercise state detected by the exercise state detection unit does not satisfy a predetermined reference.

8. The exercise promotion system according to claim 1, wherein
the work environment providing device is a display, and
the work environment change unit changes a display state of the display so that the display state of the display becomes a less appropriate state when the exercise state detected by the exercise state detection unit does not satisfy a predetermined reference.

9. The exercise promotion system according to claim 8, wherein
the display state of the display indicates at least one of a height, an elevation angle, a horizontal angle, an effective region, brightness, and a contrast ratio of an image display surface of the display.

10. A method of controlling an exercise promotion system including a work environment providing device configured to provide a work environment for improving efficiency of work of a user, the method comprising:
detecting, by the exercise promotion system, an exercise state of the user's lower limb; and
changing, by the exercise promotion system, the work environment so that the work environment deteriorates when the exercise state does not meet a predetermined reference.

11. A non-transitory computer readable medium storing a program that causes a computer to perform the method according to claim 10.
